(19) United States
(12) Patent Application Publication
(10) Pub. No.: US 2024/0298921 A1
(43) Pub. Date: Sep. 12, 2024

(54) EYEWEAR FOR COUGH

(71) Applicant: IpVenture, Inc., San Jose, CA (US)

(72) Inventors: THOMAS A. HOWELL, SAN JOSE, CA (US); C. Douglass Thomas, Saratoga, CA (US); Peter P. Tong, Mountain View, CA (US)

(21) Appl. No.: 18/666,784

(22) Filed: May 16, 2024

Related U.S. Application Data
(63) Continuation-in-part of application No. 17/407,099, filed on Aug. 19, 2021, now abandoned.

(51) Int. Cl. A61B 5/08; A61B 5/00; A61B 7/00

(52) U.S. Cl. CPC A61B 5/0823; A61B 5/6803; A61B 5/7275; A61B 7/00; A61B 2560/0223; A61B 2562/0204; A61B 2562/04

(57) ABSTRACT

Electronic eyewear for cough. In one embodiment, the eyewear includes a gyroscope to help identify a relative orientation of the eyewear. In another embodiment, the eyewear is configured to determine its absolute orientation based on, for example, calibration or a compass. In one embodiment, the eyewear includes microphones to determine location of a sound source relative to the eyewear. The sound can be from a cough. Based on relative magnitudes of the outputs of the different microphones, the eyewear can determine the location of the sound source relative to location and orientation of the eyewear. In one embodiment, the eyewear includes a database of sound sources. Based on data in the database, the eyewear can determine additional information regarding the sound, such as it being a cough and it being from the user of the eyewear. Then the eyewear can recommend to the user a course of action.

### CROSS REFERENCE TO RELATED APPLICATIONS

This application is a continuation-in-part of U.S. patent application Ser. No. 17/407,099, filed Aug. 19, 2021, and entitled “EYEWEAR WITH HEALTH ASSESSMENT, RISK MONITORING AND RECOVERY ASSISTANCE,” which is hereby incorporated herein by reference and which application claims priority benefit of: (i) U.S. Provisional Patent Application No. 63/092,142, filed Oct. 15, 2020, and entitled “EYEWEAR WITH HEALTH ASSESSMENT, RISK MONITORING AND RECORVERY ASSISTANCE,” which is hereby incorporated herein by reference; (ii) U.S. Provisional Patent Application No. 63/074,710, filed Sep. 4, 2020, and entitled “EYEWEAR WITH HEALTH ASSESSMENT, RISK MONITORING AND RECORVERY ASSISTANCE,” which is hereby incorporated herein by reference; (iii) U.S. Provisional Patent Application No. 63/073,405, filed Sep. 1, 2020, and entitled “EYEWEAR WITH HEALTH ASSESSMENT, RISK MONITORING AND RECORVERY ASSISTANCE,” which is hereby incorporated herein by reference; and (iv) U.S. Provisional Patent Application No. 63/067,383, filed Aug. 19, 2020, and entitled “EYEWEAR WITH HEALTH ASSESSMENT, RISK MONITORING AND RECORVERY ASSISTANCE,” which is hereby incorporated herein by reference.

### BACKGROUND

Today, people faced many health risks that are impacted by not only themselves but others. Health risks are impacted by geographical locations that a person visits as well by other persons that the person comes in contact with. It is, however, difficult for people to understand and monitor their health condition and health risks. As a result, there is a need for devices that can assist people and better understand not only their health condition but also health risks that their environment or others are imposing on them. One specific area of concern is coughing from ourselves and others around us.

### BRIEF DESCRIPTION OF THE DRAWINGS

The invention will be readily understood by the following detailed description in conjunction with the accompanying drawings, wherein like reference numerals designate like elements, and in which:

FIG. 1 is a diagram illustrating an operational environment for a pair of eyeglasses according to one embodiment.

FIG. 2 illustrates a block diagram of an electronic system for eyewear.

FIG. 3 is a flow diagram of a proximity monitoring process according to one embodiment.

FIG. 4 is a diagram illustrating peer-to-peer proximity monitoring according to one embodiment.

FIG. 5A illustrates a block diagram of an electronic system for eyewear.

FIG. 5B illustrates an eyewear with a plurality of microphones.

FIG. 6 is a flow diagram of a risk monitoring process according to one embodiment.

FIG. 7 is a block diagram of a risk evaluation engine according to one embodiment.

FIG. 8 is a diagram illustrating a pair of eyeglasses capable of being wirelessly coupled to a mobile phone, according to one embodiment.

### SUMMARY

Embodiments of electronic eyewear configured to provide health condition or health risk monitoring or guidance to its user are disclosed. Electronic eyewear, when being worn, is conveniently positioned to provide health condition or health risk monitoring. The eyewear can include various detectors for monitoring health conditions. The eyewear can also monitor the geographic location of the eyewear, which can impact risk level to the user. The eyewear can also measure distance to other persons for risk monitoring and/or facilitating contact tracing. The eyewear can serve to alert its user or facilitate alerting others. The user of the eyewear can configure a risk profile so that the eyewear provides customized health condition or health risk monitoring. Other embodiments can assist a user who is ill, such as, for example, by monitoring their health, monitoring for particular illnesses, tracking worsening conditions, and/or providing guidance to the user.

Embodiments of the invention can be implemented in numerous ways, including a method, system, device, apparatus (including a computer readable medium and graphical user interface). Several embodiments of the invention are discussed below.

As a system enabling a user to monitor and be informed about their health and risks thereto, one embodiment can, for example, include at least an eyewear and a software application. The eyewear can be configured to be worn on the head of the user, and the eyewear can include at least: a location detector, a temperature detector, an activity detector, a user condition detector, and a wireless communication device. The eyewear can also include a memory configured to store detector data acquired by the location detector, the temperature detector, the activity detector and the user condition detector; and a controller operatively connected to the location detector, the temperature detector, the activity detector, the user condition detector and the wireless communication device, the controller configured to receive and store the detector data to the memory. The software application can operate on a mobile computing device configured to receive the detector data wirelessly or via wire from the eyewear, process the detector data in view of user supplied data, and produce a health risk alert.

As a method for alerting a user of eyewear that they have an elevated medical risk, the eyewear including a plurality of detectors, one embodiment can, for example, include at least: capturing detector data using the detectors of the eyewear; accessing user risk data associated with the user; determining a risk level to the user of the eyewear based on the detector data and the user risk data; and notifying the user of the risk level.

As eyewear, one embodiment can, for example, include at least: a location detector, at least one infrared emitter, a plurality of infrared detectors, an image capture device, an activity detector, and a wireless communication device. The eyewear can also include a controller configured to: received and store detector data; determine whether another person is within a threshold separation distance; capture data associated with the another person if it is determined that the another person is within the threshold separation distance; and store the captured data along with related attribute data.

As eyewear, one embodiment can, for example, include at least: a location detector, a temperature detector, an activity detector, a user condition detector, and a wireless communication device. The eyewear can also include a memory configured to store detector data acquired by the location detector, the temperature detector, the activity detector and the user condition detector; and a controller operatively connected to the location detector, the temperature detector, the activity detector, the user condition detector and the wireless communication device. The controller can be configured to: receive and store the detector data to the memory; and transmit at least a portion of the detector data to another computing device for evaluation.

In one embodiment, an eyewear can be used regarding coughing of the user or people around the user. The eyewear can be used to identify its orientation relative to a preset orientation, such as by including a gyroscope. In another embodiment, the eyewear is configured to determine the preset orientation also based on, for example, a calibration process or a compass. In one embodiment, the eyewear includes a number of microphones to determine location of a sound source relative to the eyewear. The sound source can be a cough. Based on relative magnitudes of the outputs of the different microphones, the eyewear can determine the location of the sound source relative to location and orientation of the eyewear. In one embodiment, the eyewear also includes a database of sound sources. Based on data in the database, the eyewear can help determine additional information regarding the sound, such as it being a cough and it being from the user of the eyewear. Then the eyewear can recommend to the user a course of action.

Other aspects and advantages of the invention will become apparent from the following detailed description taken in conjunction with the accompanying drawings which illustrate, by way of example, the principles of the invention.

### DESCRIPTION OF EMBODIMENTS OF THE INVENTION

Embodiments disclosed herein pertain to electronic eyewear configured to provide health condition or health risk monitoring or guidance to a user of the electronic eyewear. Electronic eyewear, when being worn, is conveniently positioned to provide health condition or health risk monitoring. The eyewear can include various detectors for monitoring health conditions. The eyewear can also monitor the geographic location of the eyewear, which can impact risk level to the user. The eyewear can also measure distance to other persons for risk monitoring and/or facilitating contact tracing. The eyewear can service to alert its user or facilitate alerting others. The user of the eyewear can configure a risk profile so that the eyewear provides customized health condition or health risk monitoring. Other embodiments can assist a user who is ill, such as, for example, by monitoring their health, monitoring for particular illnesses, tracking worsening conditions, and/or providing guidance to the user.

The electronic eyewear can itself include all necessary electronic components so as to perform the health condition or risk monitoring. Alternatively or additionally, the electronic eyewear can communicate with and be assisted by storage or computing resources of one or more nearby computing device, such as a tablet computer, notebook computer, smart phone, wearable computing device, or other suitable device. Such computing devices can be wired or wirelessly attached to the electronic eyewear.

Still further, the electronic eyewear can also communication with another like electronic eyewear to share of data therebetween. The data shared can be user condition data, such as temperature. The data shared can include a risk level of a user, such as a risk that one user faces if coming in proximity to another user.

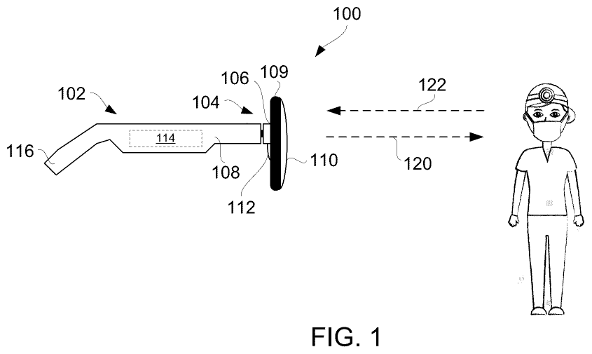

Embodiments of various aspects of the invention are discussed below with reference to FIGS. 1-8. However, those skilled in the art will readily appreciate that the detailed description given herein with respect to these figures is for explanatory purposes as the invention extends beyond these limited embodiments.

FIG. 1 is a diagram illustrating an operational environment for a pair of eyeglasses 100 according to one embodiment. More generally, the pair of eyeglasses 100 can be referred to as eyewear. The eyeglasses 100 can include a frame 102. The frame 102 can include a forward portion 104 coupled to an end piece 106 that can be coupled to a temple 108. Although FIG. 1 illustrates only one side of the eyeglasses 100, the eyeglasses 100 can also have another temple and end piece on the opposite side of the frame 102. The forward portion 104 can also include a lens holder 109 and one or more lenses 110 held in position by the lens holder 109. Additionally, the forward portion 104 can include a nose piece 112 that is typically coupled to a bridge component of the forward portion 104. Typically, the bridge component would be at a central portion of the forward portion 104. The bridge component can typically support the nose piece 112.

The temple 108 can include an electronic module 114. Typically, the electronic module 114 would be partially or completely embedded within the temple 108. The electronic module 114 would include or couple to various electronic components used to provide information to or get information from a user of the eyeglasses 100. The information can, for example, be an alert. The temple 108 can also include a temple tip 116, which can be provided at a rearward portion of the temple 108.

The eyeglasses 100 can be worn by a user (not shown). When being worn by the user, the eyeglasses 100, using the electronic module 114, can monitor either the user or other nearby other persons 118, or both. The electronic module 114 can interact with one or more electronic components, either within the electronic module 114 or elsewhere within the frame 102. In one implementation, the electronic module 114 and the electronic components can be used to monitor location and/or physical conditions of the user. In another implementation, the electronic module 114 and the electronic components can be used to monitor location and/or physical conditions of another person 118. In still another implementation, the electronic module 114 and the electronic components can be used to monitor location and/or physical conditions of both the user and another person 118. In yet another embodiment, the electronic module 114 can be used to monitor and assist a user who is ill. In any of these implementations, the data gathered regarding location and/or physical conditions concerning the user or another person 118 can be stored, examined, processed, archived or transmitted to a remote device.

In one embodiment, the electronic module 114 of the eyeglasses 110 can control an electronic component to emit electromagnetic signals 120 and can also control the same or another electronic component to receive electromagnetic signals 122. In one implementation, the signals 120 being emitted are directed in some cases towards the another person 118 and reflect back from the another person 118 to provide received electromagnetic signals 122. In another implementation, the emitted signals 120 are not needed. More generally, the electromagnetic signal can be wireless signals.

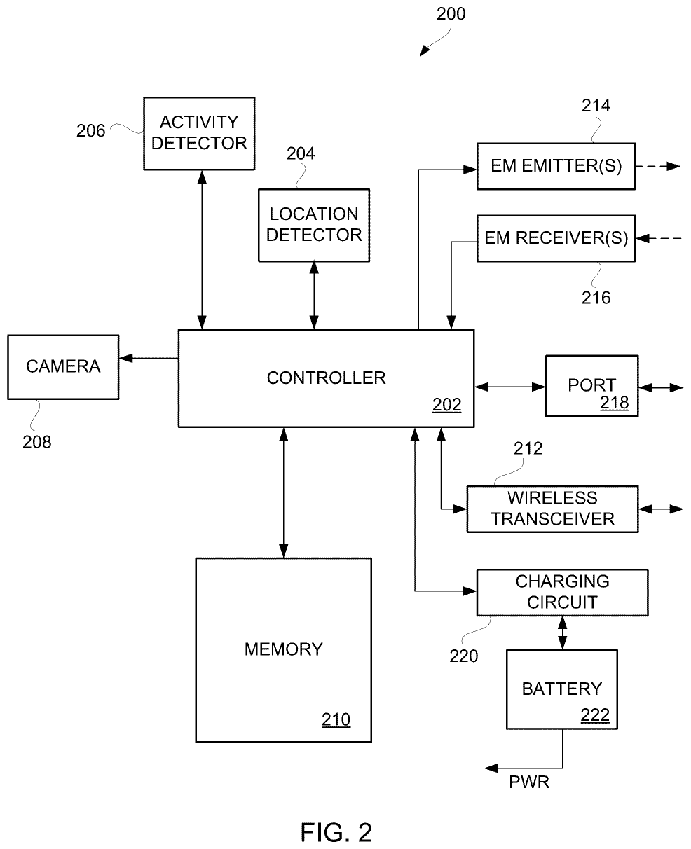

FIG. 2 illustrates a block diagram of an electronic system 200 for eyewear. For example, the electronic system 200 can be utilized in eyewear, such as the eyeglasses 100 illustrated in FIG. 1. In one implementation, the electronic system 200 can in whole or in part be included within the electronic module 114. In another implementation, the electronic system 200 is partially within the electronic module 114 and partially elsewhere within the frame of the eyewear.

The electronic system 200 can include a controller 202 that controls the overall operation of the electronic system 200. The electronic system 200 also can include a location detector 204, an activity detector 206 and a camera 208. Further still, the electronic system 200 can include one or more user condition detectors 209. The location detector 204 can pertain to one or more position determining devices that can receive wireless signals and use such to determine the location of the electronic system 200, and thus the location of the eyewear including the electronic system 200.

The activity detector 206 can detect activity associated with the electronic system 200. The activity can pertain to movement, speed, direction of travel, and the like. The camera 208 can be used to capture a digital image. The digital image can, for example, be an image of something in the user's field of view vis-à-vis the eyewear being worn by the user. For example, the digital image being captured by the camera 208 can pertain to a person that is in front of the user wearing the eyewear. In such an example, with respect to FIG. 1, the camera 208 could acquire the image of the another person 118. In another embodiment, the camera 208 (or an additional camera) could acquire an image of the user wearing the eyewear. The images can be visual or other wavelengths, such as infrared (including near-infrared radiation).

The electronic system 200 can also include a memory 210. The memory 210 can store captured data. For example, the memory 210 can store location data detected by the location detector 204, activity data acquired by the activity detector 206, and digital images taken by the camera 208. The digital images can be still images or videos. The memory 210 can also store a user profile for the user of the eyewear and/or one or more configuration settings applicable to the user of the electronic system 200.

The electronic system 200 can also include a wireless transceiver 212 that enables electronic system 202 interact with one or more wireless data networks. For example, through use of the wireless transceiver 212, the electronic system 200 can send and receive data to and from a remote computing device. The remote computing device, in one embodiment, can pertain to a mobile computing device, such as a mobile phone with application processing (i.e., smart phone), tablet computer, portable computer, and the like. The remote computing device can also be a wearable electronic device, such as another pair of electronic glasses, smart watch, fitness or health tracker, etc.

The electronic system 200 can also include one or more electromagnetic emitters 214 and one or more electromagnetic receivers 216. The electromagnetic emitters 214 can emit electromagnetic signals that can be reflected from an object (such as a person), and reflected back to the electromagnetic receivers 216. In some embodiments, the electromagnetic emitters 214 are not needed since the electromagnetic receivers 216 can receive electromagnetic radiation sent from another source or reflected or emitted from another source or object. Such signals can be used for a variety of purposes. In one implementation, the electromagnetic emitters 214 and/or the electromagnetic receivers 216 can be used to at least in part measure a temperature of the another person. In another implementation, the electromagnetic emitters 214 and the electromagnetic receivers 216 can be used to measure a distance from the eyewear to the another person. As an example, the electromagnetic signals being sent or received can be infrared radiation (including near-infrared radiation). As another example, the electromagnetic signals can be other forms of wireless signals. The other forms of wireless signals being sent or received can include, for example, ultrasonic radiation.

The electronic system 200 can also include a port 218. The port 218 can provide a wired connection port that can receive a cable so that data and/or power can be received over the resulting wired connection. The electronic system 200 can also include a charging circuit 220 and a battery 222. Since eyewear is a mobile product, the electronic system 200 within eyewear is also mobile. Hence, it is important that the electronic system 200 be powered by the battery 222. In doing so, the battery 222 will occasionally require charging by the charging circuit 220. In this case, the port 218 is often connected to a cable that provides power to the electronic system 200, and such received power can then be used by the charging circuit 220 to charge the battery 222. In an alternative embodiment, the port 218 could also support an inductive connection such that a cable is not required. In such case, the port 210 is also able to receive power from the coupled connection through inductance, and then use power wirelessly received to charge the battery 222 via the charging circuit 220.

As noted above, the electronic system 200 can include the camera 208. The camera 208 can acquire a digital image associated with another person. The digital image can be processed to determine if the digital image includes an image of a head portion of the another person. If so, the processing can examine the digital image to determine whether the another person is wearing a cover over the another person's nose and/or mouth.

As noted above, the electronic system can include the EM receiver 216 can receive electromagnetic (EM) signals from another person that is nearby and in a field of view of the EM receiver. The received EM signals can be processed to evaluate a temperature of another person. For example, if the received EM signals are infrared (including near-infrared) signals, the received EM signals can be processed to extract a temperature indication of the another person. In one implementation, an image from the camera 208 can also be used to identify another person within the image and determine a geometric position of the another position, such that the received EM signals associated with the forehead can be located and used for determining the temperature of the another person.

As noted above, the electronic system 200 can include the activity detector 206. The activity detector 206 can be provided in eyewear to measure activity of the user of the eyewear. The activity measured can pertain to mere movement, or more sophisticated movements, such as speed, acceleration, and/or direction. The activity measured can also include or permit determination of duration of movement (or non-movement) of the user of the eyewear.

As noted above, the electronic system 200 can include the location detector 204. The location detector 204 can be provided in eyewear so that the location of the eyewear, and thus the user, can be known. For the location, the eyewear can evaluate a risk associated with the user's location. For example, if a virus is understood to be more prevalent at some locations and not others, then the presence of the user at a high-risk location can be recognized. The location at which events of interest occur can also be useful and acquired and stored. For example, the time and location when the electronic system 200 recognized high-risk conditions can be noted and stored. As another example, the time and location when the electronic system 200 detected that another user was not properly social distanced with respect to the user of the eyeglasses can be noted and stored.

As noted above, the electronic system 200 can include the user condition sensor 209. The user condition sensor 209 can monitor or measure one or more physical conditions of the user. The physical conditions can include one or more of temperature, oxygen level, pulse level, cough level, breathing rate, etc.

In another embodiment, the user condition sensor 209 can monitor the user's emotion level, such as joy, sadness, fear, relaxed, angry, etc. In one embodiment, this can be done, for example, by, at least in part, monitoring the user's eyes (including, for example, eye movements), pupil size, facial expressions, and skin. In another embodiment, this can be done by, at least in part, measuring skin's conductance to determine, for example, the amount of sweat the user is producing. In still another embodiment, this can be done by, at least in part, by measuring the user's pulse and temperature.

In yet another embodiment, the user condition sensor 209 can monitor the user's electromyogram (EMG), electroencephalogram (EEG), and brain-imaging signals.

In any case, the different attributes of the user being monitored can be combined to determine various conditions regarding the user.

As noted above, the electronic system 200 also includes the wireless transceiver 212. The wireless transceiver 212 can be used to facilitate transmission of data to a remote computing device, such as a notebook computer, tablet computer, smart phone, or server computer. Any of the data captured or determined by the electronic system 200 can be transmitted to the remote computing device. Also, the data captured or determined received at the remote computing device can be further processed there. For example, the remote computing device can be configured to store data for a user, perform analytics on the data to identify trends or patterns, determine suggestions or recommendations for the user, determine whether alerts are need and to who and how the alters are to be provided, etc. Also, any data determined at the remote computing device can be transmitted back to the electronic system 200. The remote computing device can operate an application program to provide health monitoring, health risk monitoring, contact tracing, etc.

The wireless transceiver 212 can also communicate with another eyewear having an electronic system. The communication can be wireless in a peer-to-peer, Wi-Fi, Bluetooth, or various other short-range wireless networks. In doing so, the respective electronic system can send and receive various data captured or determined to one or more other near-by electronic systems. In such implementations, user condition data and location data can be shared. Such can allow for sharing of one's temperature and facilitates determination of, for example, a separation distance therebetween. Moreover, such sharing of data further allows more accurate risk level that a user faces from their environment. For example, knowing the medical conditions (e.g., temperature, oxygen level, breathing rate, cough rate, etc.) of another nearby person or more directly that person's current threat level (i.e., risk they present to others) can be used by the electronic eyewear of its user to better understand the risk they are facing. For example, the electronic eyewear of a user can understand and compute its current threat level from data captured or shared based on the situation and thus dependent on: whether there is a mask on the face of the other person, what the separation distance is, duration for which the other person was proximate thereto, whether a cough happened from the other person, whether the current location is a high risk area, etc., whereby any one or more of which can be examined and use to provide significant risk monitoring.

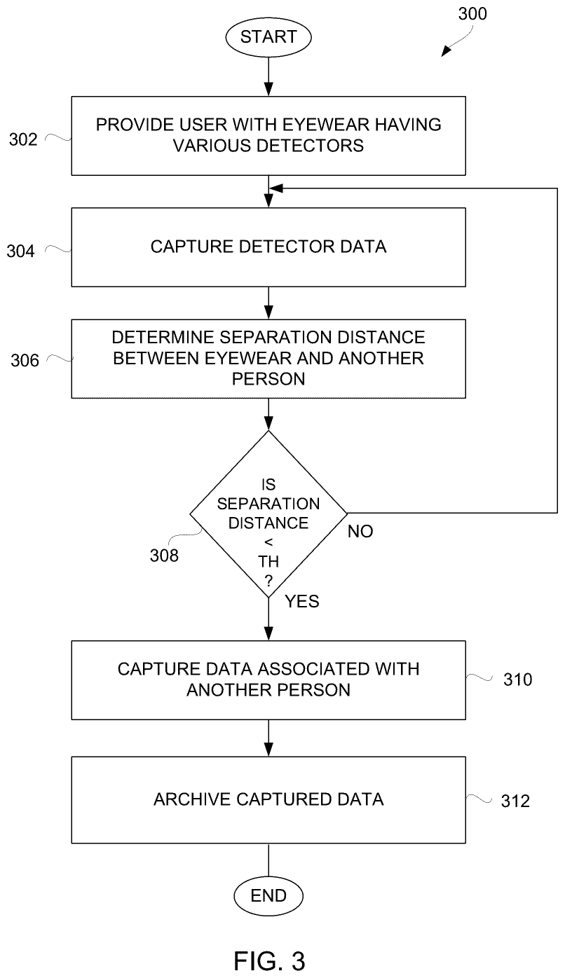

FIG. 3 is a flow diagram of a proximity monitoring process 300 according to one embodiment. The proximity monitoring process 300 can, for example, be performed by an electronic system provided within eyewear. The electronic system can, for example, be the electronic system 200 illustrated in FIG. 2.

The proximity monitoring process 300 can provide 302 a user with eyewear having various detectors. The detectors can be used to capture 304 detector data while the user is wearing the eyewear. The electronic system within the eyewear can perform various operations using the captured data. One such operation is proximity monitoring. Proximity monitoring can include detecting that others are in close proximity to the user of the eyewear.

After capturing data from the detectors, a separation distance between the eyewear and another person can be determined 306. Here, the determination 306 can make use of the captured data that has been detected by the electronic system within the eyewear. In one embodiment, the separation distance can be determined 306 based, at least in part, on electromagnetic signals. As an example, the electromagnetic signals can be received by one or more electromagnetic receivers, such as the one or more electromagnetic receivers 216 illustrated in FIG. 2. As an example, the electromagnetic signals can be infrared signals. In another embodiment, the separation distance can be determined 306 based, at least in part, on received signals from two cameras positioned on opposite ends of the eyewear's frame. In still another embodiment, other wireless devices nearby can exchange data and use such data to determine 306 a separation distance.

Next, a decision 308 can determine whether the separation distance is less than a threshold amount. The threshold amount establishes a proximity zone about the eyewear and its user. For example, the threshold amount for the separation distance can be on the order of 15 feet or less. More specifically, the threshold amount can be about 6 feet, which is a commonly suggested social distancing amount for public health concerns. The threshold amount can be set or dependent on one or more configuration settings for the electronic system within the eyewear. For example, the threshold amount can be determined from a risk level set or determined for the user. As another example, the threshold amount can be a separation distance that can be set by the user.

When the decision 308 determines that the separation distance is not less than the threshold amount, then the proximity monitoring process 300 can return to repeat the block 304 and subsequent blocks so that the proximity monitoring can continue. On the other hand, when the decision 308 determines that the separation distance is less than the threshold amount, then data associated with another person can be captured 310. In this case, the another person is deemed proximate to the user of the eyewear. The data being captured 310 can, for example, include a digital image associated with another person captured by a camera, such as the camera 208 illustrated in FIG. 2. The captured data 310 can also include a location associated with the eyewear (and likewise associated with another person whom is proximate to the user), which can be determined by a location detector, such as the location detector 204 illustrated in FIG. 2. Following the data capture 310, the captured data can be archived 312. For example, the captured data can be archived 312 in a memory, such as the memory 210 illustrated in FIG. 2. The captured data can also be archived 312 at a remote computing device or remote storage device by being transmitted, wired or wirelessly, from the eyewear to the remote computing device or remote storage device.

Following the archival 312 of the captured data, the proximity monitoring process 300 can return to repeat the block 304 and subsequent blocks so that the proximity monitoring can continue. It may be advantageous to delay for a period of time following the block 312 before proceeding to the block 304 so that data need not be repeatedly captured concerning the same proximity detection.

In some embodiments, not only does the user wear eyewear have an electronic system but also another person also wears eyewear with an electronic system. In such an embodiment, the respective electronic systems within the respective eyewear can communicate with one another. In such embodiments, a separation distance can be determined through wireless communication with another wireless device that is within wireless range.

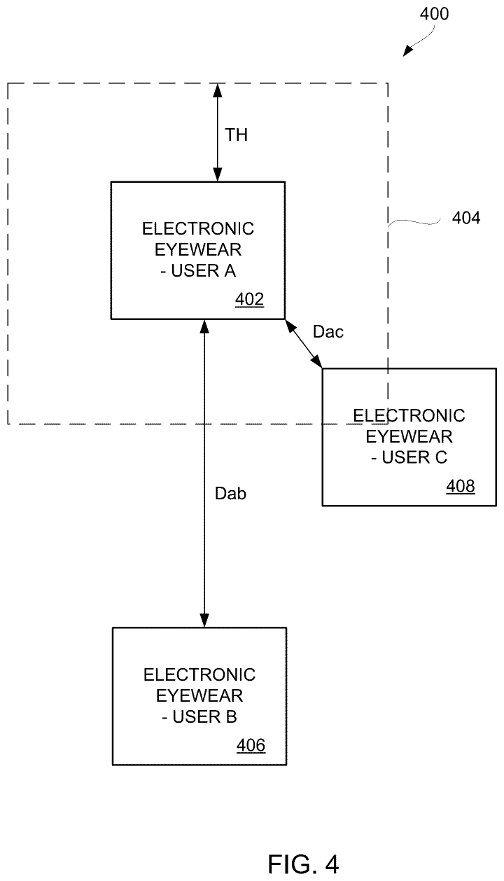

FIG. 4 is a diagram illustrating peer-to-peer proximity monitoring 400 according to one embodiment. The peer-to-peer proximity monitoring 400 illustrates a scenario in which a user A is wearing electronic eyewear 402, user B is wearing electronic eyewear 406, and user C is wearing electronic eyewear 408. The electronic eyewear 402 for the user A is configured to provide a proximity zone 404 about the electronic eyewear 402 (as well as the user A). The depth of the proximity zone 404 is a threshold amount (TH), which can be configurable, static or dynamically determined. The operation of the electronic eyewear 402 in view of the proximity zone 404 can operate to recognize the existence of the electronic eyewear 406 of the user B as well as the electronic eyewear 408 for the user C. In the example depicted in FIG. 4, the electronic eyewear 402 can determine a separation distance Dab between the electronic eyewear 402 and the electronic eyewear 406 as well as a separation distance Dac between the electronic eyewear 402 and the electronic eyewear 408. Having determined the separation distances, the electronic eyewear can then evaluate the separation distances to determine whether either the electronic eyewear 406 and or the electronic eyewear 408 are within the proximity zone 404 of the electronic eyewear 402. In this example, it is shown that the electronic eyewear 406 is outside of the proximity zone 404, while the electronic eyewear 408 is at least partially within the proximity zone 404. As such, the electronic eyewear 402 can largely ignore the electronic eyewear 406 but can capture data associated with the electronic eyewear 408 and/or its user C. As discussed here, data can be captured using electronic components of the electronic eyewear 402, or can be captured using electronic components of the electronic eyewear 408 and then shared (e.g., wirelessly) with the electronic eyewear 402, or some combination thereof. The peer-to-peer proximity monitoring 400 allow for contact tracing, medical risk monitoring, etc., often with additional computing resources of another mobile or wearable electronic device (e.g., mobile phone with application processing capabilities, also known as a smart phone).

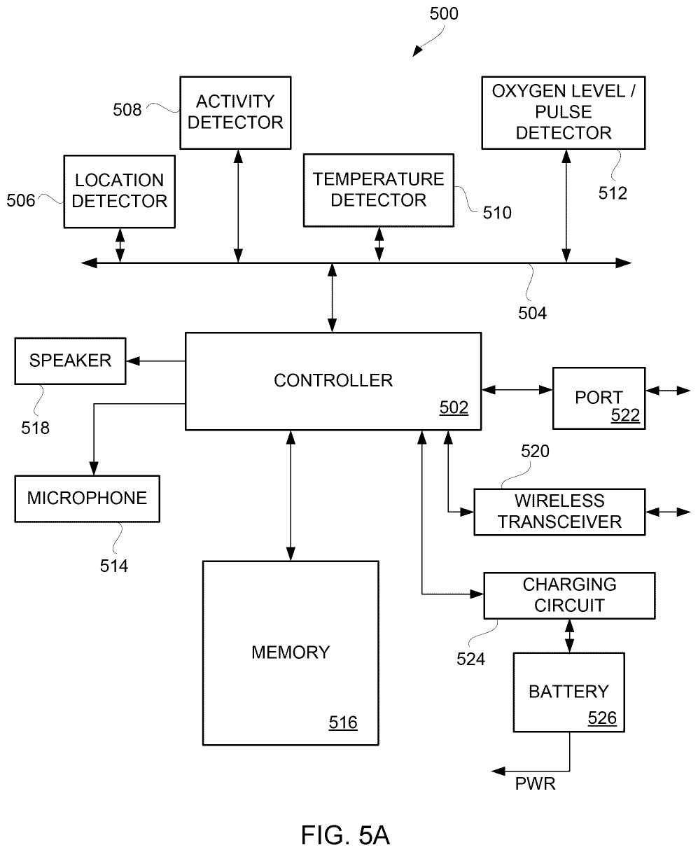

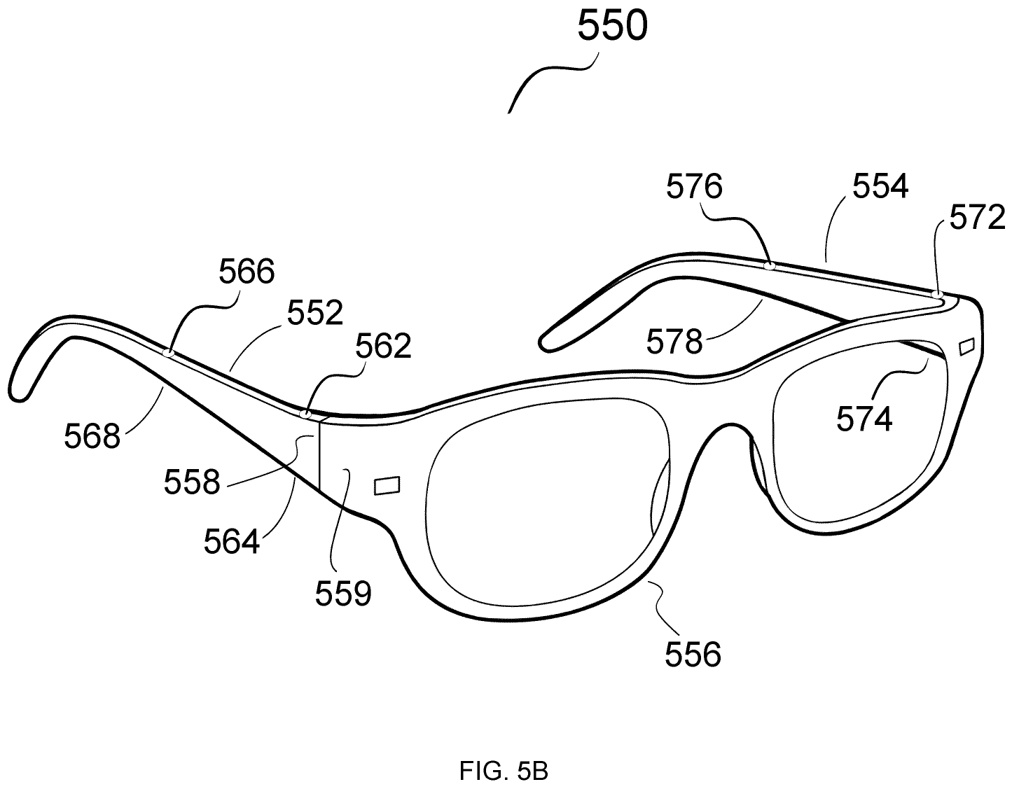

FIG. 5A illustrates a block diagram of an electronic system 500 for eyewear. For example, the electronic system 500 can be utilized in eyewear, such as the eyeglasses 100 illustrated in FIG. 1. In one implementation, the electronic system 500 can in whole or in part be included within the electronic module 114. In another implementation, the electronic system 500 is partially within the electronic module 114 and partially elsewhere within the frame of the eyewear.

The electronic system 500 can include a controller 502 that controls the overall operation of the electronic system 500. In general, it should be understood that the controller 502 can include one or more electronic circuits and/or integrated circuits. The electronic system 500 can also be coupled to one or more detectors. The one or more detectors can be within the eyewear, such as embedded within the eyewear. Alternatively, it is possible that one or more of the detectors can be external to the eyewear but proximate thereto and communicate wirelessly with the electronic system 500 provided within the eyewear. As shown in FIG. 5A, the electronic system 500 can include a data bus 504. The various detectors can couple to the data bus 504 to communicate with the controller 502. The various detectors can include a location detector 506, an activity detector 508, a temperature detector 510, and an oxygen level/pulse detector 512.

The location detector 506 can pertain to one or more position determining devices that can receive wireless signals and use such to determine the location of the electronic system 500, and thus the location of the eyewear including the electronic system 500. The activity detector 508 can detect activity associated with the electronic system 500. The activity can pertain to movement, speed, direction of travel, and the like. The temperature detector 510 can detect the temperature of the user of the eyewear. The oxygen level/pulse detector 512 can be used to measure oxygenation (or oxygen level) or pulse of the user of the eyewear. Additionally, the electronic system 500 can also include a microphone 514 that can couple to the controller 502. The microphone 514 allows audio pickup in the vicinity of the eyewear.

The electronic system 500 can also include a memory 516. The memory 516 can store captured data. For example, the memory 516 can store location data detected by the location detector 506, activity data acquired by the activity detector 508, temperature data acquired by the temperature detector 510, and oxygen level or pulse data acquired by the oxygen level/pulse detector 512. The memory 516 can also store risk data for the user of the eyewear. The memory 516 can also store a user profile for the user of the eyewear and/or one or more configuration settings applicable to the user of the electronic system 500. For example, one of the configuration setting can be a risk level setting for the user. As another example, one of the configuration settings can be a separation distance to be maintained and monitored.

Still further, the electronic system 500 can also include one or more speakers 518. The one or more speakers 518 can be connected to the controller 502. The one or more speakers 518 can be used to output audio (e.g., audio alerts) to the user of the eyewear.

The electronic system 500 can also include a wireless transceiver 520 that enables the electronic system 500 interact with one or more wireless data networks. For example, through use of the wireless transceiver 520, the electronic system 200 can send and receive data to and from a remote computing device. The remote computing device, in one embodiment, can pertain to a remote computing device, such as a mobile phone, tablet computer, portable computer, and the like. The remote computing device can also be a wearable electronic device, such as another pair of electronic glasses, smart watch, smart phone, fitness or health tracker, etc.

The electronic system 500 can also include a port 522. The port 522 can provide a wired connection port that can receive a cable so that data and/or power can be received over the resulting wired connection. The electronic system 500 can also include a charging circuit 524 and a battery 526. Since eyewear is a mobile product, the electronic system 500 within eyewear is also mobile. Hence, the electronic system 500 can be powered by the battery 526. In doing so, the battery 526 will occasionally require charging by the charging circuit 524. In this case, the port 522 is often connected to a cable that provides power to the electronic system 500, and such received power can then be used by the charging circuit 524 to charge the battery 526. In an alternative embodiment, the port 522 could also support an inductive connection such that a cable is not required. In such case, the port 522 is also able to receive power from the coupled connection through inductance, and then use power wirelessly received to charge the battery 526 via the charging circuit 524. Additionally, the electronic system 500 could also include or couple to a solar panel as another means to provide power to the electronic system 500.

The electronic system 500 can also include any of the components included within the electronic system 200 illustrated in FIG. 2.

As noted above, the electronic system 500 can include the microphone 514. In one implementation, the microphone 514 can be implemented by a plurality of microphones. The microphone 514 can provide audio pickup. The audio pickup could be from the user, and be used to detect coughing, difficulty breathing, etc. This permits the monitoring of cough rate, breathing rate, etc. The audio pickup could also be used to detect whether the user is in a crowed area, such as by detecting different voices in the vicinity.

As noted above, the electronic system 500 can include the temperature detector 510. The temperature detector 510 can be provided in eyewear so as to couple against (or nearly so) a user's head, e.g., temple or nose of the user. Here, the temperature detector 510 can measure the temperature of the user of the eyewear. In one implementation, the temperature detector 510 can be implemented as a photodiode, IR sensor, or thermistor.

As noted above, the electronic system 500 can include the activity detector 508. The activity detector 508 can be provided in eyewear so measure activity of the user of the eyewear. The activity measured can pertain to mere movement, or more sophisticated movements, such as speed, acceleration, and/or direction. The activity measured can also include or permit determination of duration of movement (or non-movement) of the user of the eyewear.

As noted above, the electronic system 500 can include the oxygen level/pulse detector 510. The oxygen level of the user pertains to oxygenation or oxygen saturation of the user blood, and the pulse pertains to a heat rate for the user. The oxygen level/pulse detector 512 can be provided in eyewear so as to couple against (or nearly so) a user's head, e.g., temple or nose of the user. In one implementation, the oxygenation/pulse detector 512 can be implemented as a pulse oximeter.

As noted above, the electronic system 500 can include the location detector 506. The location detector 506 can be provided in eyewear so that the location of the eyewear, and thus the user, can be known. For the location, the eyewear can evaluate a risk associated with the user's location. For example, if a virus is understood to be more prevalent at some locations and not others, then the presence of the user at a high-risk location can be recognized. The location at which events of interest occur can also be useful and acquired and stored. For example, the time and location when the electronic system 500 recognized high-risk conditions can be noted and stored.

FIG. 5B illustrates an eyewear 550 with a plurality of microphones that can be used to determine the source of a cough or sneeze. In one embodiment, the eyewear 550 includes a front portion 556, which includes a lens holder. The front portion 556 is coupled to two side portions, arms or temples; and they are a right one 552 and a left one 554.

Each arm can have one or more microphones. For example, the right arm 552 includes one or more microphones 562, 564, 566, and 568. Microphones 562 and 564 can be closer to the front portion 556 than microphones 566 and 568. For example, microphones 562 and 564 can be adjacent to a hinge 558. In another example, microphones 562 and 564 can be adjacent to the hinge 558 but in the front portion 556, such as in a right end region 559 of the front portion 556, with one on top and the other one at the bottom. The two microphones 566 and 568 can be close to the right ear of the user, such as in front of the right ear.

Microphones 562 and 566 can be above microphones 564 and 568 respectively. For example, microphones 562 and 566 can be on the top part of the arm 552, while microphones 564 and 568 can be on the bottom part of the arm 552. In one embodiment, with respect to the arm 552, microphone 562 can be directly above microphone 564, and microphone 566 can be directly above 568.

Similarly, there can be one or more microphones in the arm 554. For example, the left arm 554 includes one or more microphones 572, 574, 576, and 578. Microphones 572 and 574 can be closer to the front portion 556 than microphones 576 and 578. For example, microphones 572 and 574 can be adjacent to a left hinge. In another example, microphones 572 and 574 can be adjacent to the left hinge but in the front portion 556. Microphones 572 and 576 can be above microphones 574 and 578 respectively. For example, microphones 572 and 576 can be on the top part of the arm 554, while microphones 574 and 578 can be on the bottom part of the arm 554. In one embodiment, with respect to arm 554, microphone 572 can be directly above microphone 574, and microphone 576 can be directly above 578.

In one embodiment, the eyewear 550 shown in FIG. 5B can be used to determine the source of coughing or sneezing. For example, when a person close to the eyewear sneezes, one or more of the microphones can have a higher signal than other microphones. Based on the relative values of the outputs of the different microphones, one or more processors in the eyewear can determine the direction where the sneeze comes from relative to the eyewear, such as relative to the eyewear's location and orientation.

To illustrate, if the user wearing the eyewear sneezes, microphones 564 and 574 can have a higher signal than microphones 562 and 572. In one embodiment, the microphones 564 and 574 may even saturate or may peak. Typically, if a microphone saturates, the output from the microphone can stay at the saturated voltage for a duration of time, such as between 50 milliseconds and 200 milliseconds, depending on the designs of the microphone circuits. In another embodiment, the eyewear 550 does not include microphones 572 and 574. Then the user sneezing can be determined based on the microphone 564 having a higher output than the microphone 562.

As explained in other areas of the specification, for example, the eyewear can include a database configured to store attributes or signatures of different types of sounds. The different types of sounds can include sounds of a cough, a sneeze, a cough from the user, a sneeze from the user, and other types of sound. Other types of sounds can include the sound of someone speaking, such as speaking a language.

In one embodiment, outputs from the microphones can be analyzed, such as compared with the types of sound in the database of sounds in the eyewear to identify the outputs as a cough, a sneeze, and/or as such from the user. In one embodiment, the eyewear 550 has one microphone, such as microphone 564. Its output can be analyzed based on sound signature of the user to determine whether the user coughing or other people coughing, or to determine a probability of whether the user coughing or other people coughing. Typically, more microphones can provide more accurate determination than fewer microphones.

As another example, if a person directly before the user wearing the eyewear sneezes, the one or more forward microphones, such as 562, 564, 572, and 574, can have a higher output, such as higher output voltages, than the one or more microphones at the back, such as 566, 568, 576, and 578. If a person to the right of the user sneezes, the one or more microphones on the right-hand side, such as microphones 562 and 564, 566, and 568, can register a higher output than their corresponding one or more microphones on the left-hand side, such as microphones 572, 574, 576, and 578.

In one embodiment, the eyewear 550 includes 4 microphones, namely 562, 564, 572, and 574. These 4 microphones can be used to determine if the user is coughing or sneezing relative to other sounds in the surrounding environment, such as door slam, dog bark, and hand clap.

In another embodiment, the eyewear 550 includes 6 microphones, namely 562, 564, 572, 574, and 568 and 578; and the eyewear can be used to distinguish if the user or someone else is coughing with the direction of the person coughing relative to the eyewear also determined.

In one embodiment, an eyewear can include at least a microphone, a controller and a storage medium storing data including data regarding different types of sound. In one embodiment, the controller can analyze at least the sound received by the microphone with sound in the storage medium. In one embodiment, based at least on the sound received substantially matching the sound of the user's cough in the storage medium, the controller can determine that the sound received is the user's cough. The controller can generate a recommendation for the user based on the determination. In another example, based on the determination, the controller can send a signal wirelessly to another electronic device. The another electronic device can be a mobile phone of the user, and the signal can be based on a short-range wireless network, such as a Bluetooth network. The another electronic device can analyze the signal received and determine a course of action based on the analysis, as discussed in other parts of the specification.

In one embodiment, an eyewear worn by a user to determine the direction of the sneeze can depend on an orientation of the eyewear. In one approach, the eyewear 550 can include at least a gyroscope to help identify a rotation and a tilt of the eyewear or the head of the user when the user is wearing the eyewear. Such identification can be relative to a preset rotation and relative to a preset tilt. In another embodiment, the eyewear 550 can include at least an accelerometer for determining left/right motion and/or acceleration to help identify the rotation of the head or the corresponding eyewear worn by the user; and the eyewear 550 can include another accelerometer for up/down motion and/or acceleration to help identify the tilt of the eyewear or the corresponding head.

In one embodiment, the eyewear 550 can include a compass to help determine the preset rotation and/or tilt.

In another approach, the preset rotation and/or tilt can be determined based on a calibration process. For example, the preset rotation and/or tilt can be where the user typically positions her head. The controller in the eyewear can assume that the user keeps her head pointing in one direction most of the time, such as looking straight in front. And the eyewear can measure outputs of the gyroscope in it over a long period of time and calculate the average the outputs, assuming the output being the orientation of the user wearing the eyewear looking straight in front. In one approach, similar process can be used to determine the preset tilt.

In another calibration process, the user can put on the eyewear and can be told, such as by the eyewear, to look straightly in front, without tilting up or down. A controller in the eyewear can access the outputs of a gyroscope in the eyewear and set the outputs to indicate the corresponding orientation and tilt of the head.

In one embodiment, to determine the direction of a sound, a processor in the eyewear 550 first can determine the orientation and tilt of the head. Then based on the relative magnitudes of the outputs of the microphones, the processor can determine the direction of the sound. Based on information in a database of sound in the eyewear, the processor can also identify the sound to be a cough or a sneeze or other type of sound.

A number of embodiments have been described regarding the eyewear 550 to help identify the location of a sneeze or cough. In another embodiment, such an eyewear can be used to help identify the location of a sound source, such as someone speaking. In yet another embodiment, the eyewear 550 can provide differential treatments to sound sources from different directions. For example, the head is pointing in a first direction, and the eyewear can amplify sound from the first direction more than from other directions. Or microphones in the eyewear can be configured to be more sensitive to sounds from the first direction.

In one embodiment, the eyewear 550 can be a headset.

Different embodiments described herein can be implemented using at least a headset, or an electronic apparatus worn by a user in the vicinity of the user's head. For different embodiments, the headset can be an earbud, or a pair of earbuds.

In yet another embodiment, the eyewear 550 and/or other eyewear described herein can work with one or more eyewear in its vicinity to identify the location of a sneeze, cough or other sound sources.

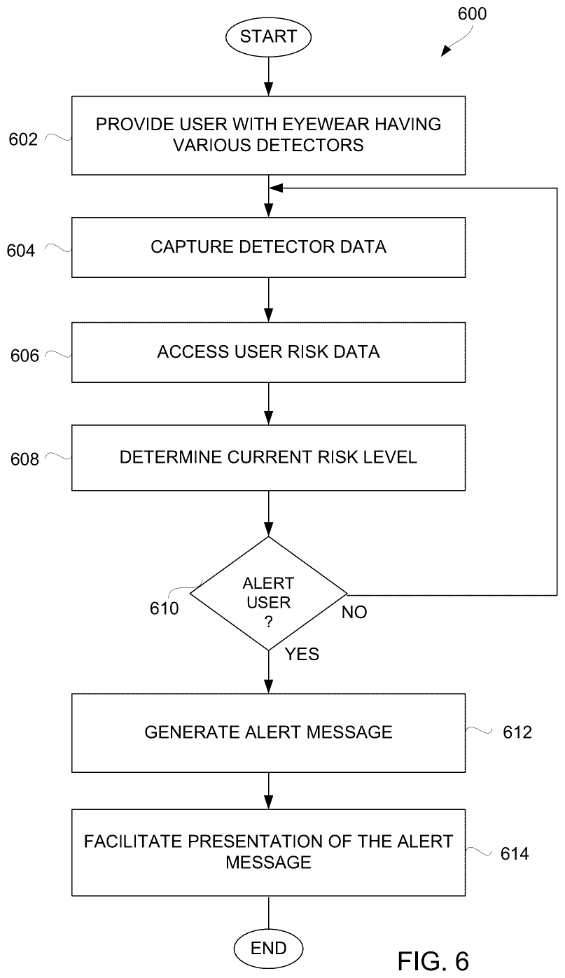

FIG. 6 is a flow diagram of a risk monitoring process 600 according to one embodiment. The risk monitoring process 600 can, for example, be performed by an electronic system provided within eyewear. The electronic system can, for example, be the electronic system 500 illustrated in FIG. 5A.

The risk monitoring process 600 can provide 602 a user with eyewear having various detectors. The detectors can be used to capture 604 detector data while the user is wearing the eyewear. The electronic system within the eyewear can perform various operations using the captured data. One such operation is risk monitoring. Risk monitoring is detecting a risk level that the user of the eyewear faces. The risk level can be based on one or more of location of the eyewear and its user, other persons nearby, health infirmities of the user, risk profile of user, etc.

After capturing data from the detectors, user risk data can be accessed 606. The user risk data can be previously stored in memory of the electronic system within the eyewear. In one implementation, the user risk data is at least in part customized to the user of the eyewear. For example, a user having poor health conditions, the risk monitoring by eyewear can be tailored to be more risk adverse. In one implementation, the user can configure a risk profile so that the risk monitoring is tuned to their risk profile.

Next, a current risk level can be determined 608. The current risk level can be determined based on the captured data as well as the user risk data. Here, the current risk level can be based on one or more of location of the eyewear and its user, other persons nearby, health infirmities of the user, risk profile of user, etc. Typically, the current risk level would then be stored to the memory within the eyewear.

After the current risk level has been determined 608, a decision 610 can determine whether the user of the eyewear should be alerted. When the decision 610 determines that the user needs not be alerted, then the risk monitoring process 600 can return to repeat the block 604 and subsequent blocks so that risk monitoring can continue. On the other hand, when the decision 610 determines that the user is to be alerted, then an alert message can be generated 612. The alert message can advise the recipient, which is often the user, to take note of their risk level and/or to consider a recommendation being provided so that they may mitigate their risk. Although the alert is well-suited to be provided to the user, the alert can alternatively or additionally be provided to another user, such as a care provider. In any case, after the alert message is generated 612, the risk monitoring process 600 can facilitate 614 presentation of the alert message. The alert message can be presented for the user, for another person, or for both. Following the block 614, the risk monitoring process 600 can return to repeat the block 604 and subsequent blocks so that risk monitoring can continue.

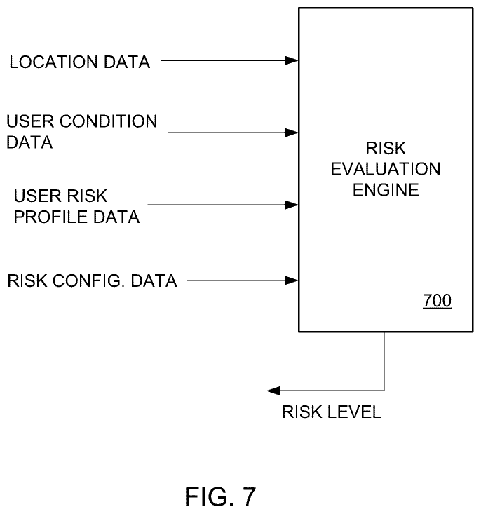

FIG. 7 is a block diagram of a risk evaluation engine 700 according to one embodiment. The risk evaluation engine 700 can operate to determine a risk level for a user of eyewear. The risk evaluation engine can, for example, be implemented by an electronic system provided within eyewear, such as the electronic system 500 illustrated in FIG. 5A. Alternatively, the risk evaluation engine can be implemented by a computing device that is in data communication with the eyewear. As one example, the risk evaluation engine can operate on a mobile phone operating a program that implements the risk evaluation engine.

As illustrated in FIG. 7, the risk evaluation engine 700 can receive a variety of different data that can be processed to generate a risk level. As illustrated in FIG. 7, the variety of different data that can be received by the risk evaluation engine 700 can make use of one or more of location data (such as location history), user condition data, user risk profile, and risk configuration data. Using the different sources of data, the risk evaluation engine 700 can determine a risk level for a user. The risk level can depend upon the user themselves, such as their risk profile and/or configuration settings. The risk level that is generated by the risk evaluation engine 700 can be subsequently used to generate a risk alert for the user.

The risk evaluation engine 700 can operate to dynamically update a risk level faced by the user. For example, the duration of time a user is at a high-risk location on a given day, week or month can cause the risk level to trend higher, as the duration increases. An increase in the user temperature might indicate that the user might be more susceptible to risks and cause the risk level to trend higher.

Embodiment can also use Artificial Intelligence (AI) to evaluate one's risk level. An AI platform can use a neural network that has been trained to look for changes, patterns or profiles of user who have a medical condition. Then, by sharing data from electronic eyewear of various users, computing devices or a central computing system can use the AI to detect or predict medical conditions, high risk levels, contact tracing, etc.

Various different embodiments for electronic eyewear can offer the following features:

### Resting Heart-Rate and Oxygen-Level

This can be done, for example, by a pulse oximeter, with at least a red LED, an infrared LED and a photodiode. The pulse oximeter can be implemented at a nose pad of the eyewear.

### Temperature Measurement

This can be done, for example, by a near-infrared photodiode in the eyewear pointing at the skin of the user, such as pointing at the user's forehead or an eye of the user. Another approach is to use a thermistor touching the skin of the user. For example, the thermistor can be at a nose pad touching the nose of the user.

### Activity Level Measurement

Activity level can be measured, for example, based on an accelerometer in the eyewear.

### Coughing Frequency Measurement

Coughing frequency can be measured using, for example, a microphone in the eyewear. In one approach, the audio sound by the user can be compared to a database of similar coughing sound to identify the audio sound as a cough. The comparison can be performed in another electronic device, such as a mobile phone, wirelessly connected to the eyewear.

In one approach, authentic coughs and other types of sound made by humans can be collected to teach a computing apparatus how to tell the difference between the two. In another approach, AI techniques can be used, such as generative adversarial networks, which can create realistic coughing, or transfer learning, where a model trained to detect one cough could train a new model to detect similar coughs.

### Shortness of Breath Measurement

One way to identify shortness of breath is via a microphone at the eyewear measuring the breathing rate of the user. Depending on age, the normal respiration rate for an adult at rest is about 12 to 20 breaths per minute. If the user has not been exercising for at least a certain amount of time, such as 5 minutes, but the breathing rate exceeds the normal respiration rate at the age of the user by a certain percentage, such as over 25%, the user can be having shortness of breath. The eyewear could alert a person of interest.

The normal respiration rate of the user can be identified by the eyewear monitoring the user.

In one approach, breathing sound can be identified by the microphone based on techniques similar to identifying the user's coughing, as described above.

In another embodiment, the eyewear can identify worsening shortness of breath or difficulty in breathing. For example, the user could have certain degree of shortness of breath or difficulty in breathing before getting the disease. The eyewear keeps track of the certain degree. After getting the disease, such as COVID-19, if the situations have worsened, such as the shortness of breath has worsened, the eyewear could alert a person of interest.

In yet another embodiment, heart rate and activity level can also be monitored.

As an example, assume the user is at rest, and the user's heart rate exceeds a normal rate, such as more than 90 times per minute, and the user also has shortness of breath. At that point, the user's health due to a disease, such as COVID-19, might have significantly deteriorated. Medical personnel might need to be quickly notified. The eyewear could alert the user and send a wireless signal to the medical personnel.

The normal heart rate of the user can be identified by the eyewear monitoring the user.

### Skin Resistance Measurement

In one embodiment, an eyewear includes at least a sensor that can be used to measure resistance of a user's skin. This can be used to determine, for example, galvanic skin response. The sensor can be used, for example, to indicate a stress level of the user. In one approach, the sensor can include two electrodes that touch the skin of the user when the eyewear is worn and can measure the resistance between the two electrodes. To illustrate, if the user sweats, which can be due to stress or physical activity or illness, the resistance between the two electrodes can decrease. In one implementation, the sensor can be located at a nose pad of the eyewear. In one embodiment, the electrodes can be plated with nickel, or other materials, such as gold, to help protect the electrodes from corrosion, such as by sweat.

### Distance Measurement

One way to measure a person a few meters away from a user can be based on two visual-light cameras and a near-infrared camera. The two visual-light cameras should be positioned far apart from each other in the eyewear, such as each being positioned close to opposite temples or end-pieces of the eyewear. The visual-light cameras can be CCD chips. Resolution of the images captured could be very low, such as 100 pixels by 100 pixels. Assuming an object is approaching the electronic eyewear, the near-infrared camera can roughly estimate the temperature of the object. If the temperature is roughly within the range of the temperature of a human, the two visual-light cameras can then take images of the object. Based on image processing, a rough identification regarding the head of the person can be made. The images of the head by the two visual-light cameras are slightly off from each other, because they measure the head from different positions. The distance of the head from the visual-light cameras can be calculated based on the concept of parallax and the differences between images from the cameras.

In the above approach, instead of two visual-light cameras and a near-infrared camera, the eyewear could have two near-infrared cameras instead. The near-infrared cameras operate can provide both rough temperature measurements and distance measurements.

Another way is for the eyewear to broadcast a message ultrasonically. As an example, an ultrasonic frequency can be 30 KHz. If someone wearing a similar eyewear is within a certain distance, that eyewear would get the message and respond automatically. Based on time elapsed between sending the message and receiving the response, the user's eyewear can determine how far the other person is away from the user. If the other device is within a certain distance, such as two meters, the user's eyewear could alert the user, such as emitting an audible beep or a vibration.

In one approach, the eyewear could broadcast the ultrasonic message every few seconds, such as every ten seconds. In another approach, the eyewear could broadcast Bluetooth signals. If someone wearing a similar eyewear is within the reception range of the signals, that eyewear would generate a response automatically. When the user's eyewear gets the response, the user's eyewear could send out the ultrasonic message every few seconds to identify the distance of the approaching eyewear. This approach could reduce power consumption of the user's eyewear.

In yet another approach, the power of the Bluetooth signals can be controlled so that the reception range of the signals is a few meters, such as two meters. In this approach, when the user's eyewear gets the response, the user's eyewear is aware of another person being in close proximity.

In yet another approach, distance measurements could be based on Bluetooth signals. The eyewear could broadcast a Bluetooth message (with a signature) periodically at a certain level, such as every 30 seconds. If someone wearing a similar eyewear is within the reception range of the signals, that other eyewear would generate a response automatically. When the user's eyewear gets the response, the user's eyewear could increase the frequency of broadcasting Bluetooth messages, such as every 50 milliseconds, but at different power levels. For example, after getting the response, the eyewear could send out another Bluetooth message at a lower level, such as ½ the power of the previous signal. This process of reducing the power level continues till the eyewear does not get a response. At that point, the signal-to-noise ratio of the Bluetooth message received by other eyewear has reached a preset level. Based on the transmitted power at that point, the distance between the two eyewear can be estimated.

### Temperature of an Approaching Person Measurement

Temperature could be measured based on a near-infrared camera and a visual-light camera, both measuring an eye of an approaching person not bearing glasses. Otherwise, the two cameras would measure the forehead of the person. The temperature of the person can be estimated by the differences from the outputs from the two cameras. Multiple readings could be taken, and the temperature of the approaching person can be based on averaging the readings.

In one approach, after determining that a person is within a few meters from the user, such as two, the eyewear would perform the temperature measurement of the person, as described above.

### Digital Assistant

The eyewear could include a digital assistant that could assist the user. The digital assistant can be based on voice recognition.

In one embodiment, a digital assistant in the eyewear can engage in simple dialogues with the user. This could be initiated by the user, such as by the user pushing a button at the eyewear and starting to talk to the digital assistant. For example, the user could say, “I don't feel good.” The assistant can then respond open-endedly, such as, “Why don't you feel good?” Through the dialogue, the assistant could spot patterns in the user's spoken language.

For example, once a day, or several times a day, the assistant could ask the user how the user is feeling, and could offer choices for the user to select to respond, such as lonely, sad, or depressed. Then the digital assistant can recognize words and phrases in subsequent dialogues with the user that could be contextually likely to be associated with the selected choice. In one embodiment, the assistant could provide some simple counseling based on standard cognitive behavioral therapy. In another embodiment, if the assistant decides that the user is depressed, the assistant could suggest activities the user likes that could enhance the user's mood or distract the user. For example, the assistant could play some music the user likes or calming music.

In one embodiment, the digital assistant could be trained to analyze (a) syntactic patterns in the user's words, such as the frequency of nouns used versus adjectives; (b) classes of words the user uses, such as whether they are more related to perception or action; (c) how often the user talks to the digital assistant; (d) whether the user changes topics more abruptly than the user's average responses; and (e) acoustic features, such as changes in volume, pitch, and frequency of pauses by the user. Based on such analysis, the assistant could determine, for example, a potential mental state of the user.

In one embodiment, the digital assistant could identify dangerous signs. For example, by analyzing dialogue from the user for an extended period of time, the assistant could identify a drastic change in the user's verbal communication. Note that the analysis could be done by a remote device, based on the user's daily communication data transmitted by the eyewear. Or, in another embodiment, the analysis, or a portion of the analysis, could be done by the eyewear. The analysis could be based on machine learning. In view of the dangerous signs identified, in one embodiment, a professional operator or a close relative of the user could be alerted, and this could be done, for example, by the digital assistant.

One embodiment for voice recognition could use processors or servers remotely accessed via the Internet (such as in the cloud), such as a GPU, to train voice recognition models, which can be a machine-learning-driven task. And the embodiment could use a lower power processor, a client processor, or an edge processor in the eyewear to run inferences. In one embodiment, with the models trained, the client processor could run voice recognition without the need to wirelessly connect to remote servers. This could lead to consuming less power, reducing latency, and/or giving a better user experience. In another embodiment, the eyewear could directly interact with remote server(s) without using a client processor.

In one embodiment, for the client processor, the eyewear could use a lighter-version TPU (Tensor Processing Unit), a custom chip optimized for inference in edge devices. Instead of using TensorFlow, a Google machine learning development platform, other embodiments could use development platforms, such as Caffe2 or PyTorch. In one embodiment, the eyewear can include an Amazon's AZ1 Neural Edge processor, which at least can help answer questions via Amazon's Alexa, based on, for example, neural speech models to process audio by the eyewear. There could be on-device memory to help voice operations or natural-language processing.

In one embodiment, different embodiments could train and/or use machine learning models in remote servers for other applications, such as image recognition.

### Calculate a Risk Index

One approach to use some or all of the above measurements is to use them to calculate an infection probability or a risk index. Based on the number, the user can get an indication as to her chance of being infected, and decide if she should go to a medical facility to be tested for a corresponding illness.

### Keep Track of the Identity of an Approaching Person

One way is based on the approaching person also wearing a similar eyewear. The two pairs of eyewear could ping each other wirelessly and exchange identification signals if they are within a certain distance, such as two meters, from each other for more than a preset amount of time, such as 2 minutes. The process can be via Bluetooth network. The user's electronic eyewear can keep a record of the encounter.

Another way can be anonymous. For example, the user's eyewear can keep track of the received identification signals, and check them against the publicized signals of individuals known to be infected.

### Additional Aspects

A number of approaches have been described using near-infrared cameras. In other embodiments, infrared cameras can be used instead.

In one embodiment, the eyewear is also applicable to help the user when the user is ill, such as the user has COVID-19. For example, a digital assistant in the eyewear or coupled to the eyewear could respond request for assistance from the user. If the request for assistance is verbal, then the responses can be standardized or a most appropriate response can be selected by natural processing and/or artificial intelligence. The responses can depend on health condition of the user that are monitored by the eyewear.

In one embodiment, if it is known that the user is sick due to a specific disease, and it is a disease with at least some known characteristics. The eyewear can include a database or couple to a remote database. The database can include at least the known characteristics of the specific disease. The eyewear could keep track of the user's conditions, as measured by different detectors, such as the detectors discussed herein. The eyewear by itself or by an application operating on a remote computing device coupled thereto, or by both can compare tracked user conditions with information in the database. If the user's conditions worsen, particularly if the user's conditions worsen relative to the characteristics of the disease in the database, the eyewear and/or an application operating on a remote computing device coupled thereto could alert medical personnel, and may provide the user's condition to the medical personnel.

The eyewear, or an application operating on a remote computing device coupled thereto, could periodically give the user some moral support or encouragement or a medical update. For example, a digital assistant can periodically engage in dialogues with the user, particularly if the sickness requires isolation. The digital assistant could ask the user if the user wants to hear a song that the digital assistant knows the user likes, based on information in a user's profile previously provided by the user. If the user's response is yes, the digital assistant could play the song via the eyewear for the user.

As another example, the assistant could remind the user with recommendations or guidance to help the user get better, such as the user should get some sun during the day. The eyewear could periodically ask the user if the user has been drinking water, if the disease requires the user to be regularly hydrated.

As still an example, if the user has been tested positive for a particular illness, such as the COVID-19 virus, the eyewear, or an application operating on a remote computing device coupled thereto, could provide standard information regarding the particular illness, such as information from CDC (Centers for Disease Control and Prevention). The user could ask the digital assistant questions regarding the particular illness. If the database has answers to the questions, the digital assistant could respond accordingly. For example, the user could ask the digital assistant how many days a family member of an infected person should be quarantined from other family members. As another example, if the user has increased coughing, but much lower than a critical level, the eyewear could identify such changes, and suggest the user to drink a cup of warm honey.

The eyewear could keep monitoring the different attributes of the user. Based on available medical information, some major concerns may include worsening shortness of breath or difficulty breathing, persistent pain or pressure in the chest, new confusion, bluish lips or face, or drop in oxygen level in the blood. Examples of techniques for the eyewear to help identify shortness of breath and oxygen level have been explained in this application. Examples of techniques for the eyewear, or an application operating on a remote computing device coupled thereto, to help identify persistent pain or pressure in the chest, confusion, and bluish lips or face will be explained. If the eyewear has identified one or more of major concerns, the eyewear, or an application operating on a remote computing device coupled thereto, could alert medical personnel.

In one embodiment, a digital assistant in the eyewear, or an application operating on a remote computing device coupled thereto, could ask the user if the user feels tightness or pressure or pain in the chest. If the user's respond affirmatively, the eyewear could periodically, like every 2 hours, ask the user again. If the tightness does not disappear within a specific period of time, such as 24 hours, or if the tightness or pain worsens, the eyewear, or an application operating on a remote computing device coupled thereto, could contact medical personnel. In another embodiment, the user can let the eyewear, or an application operating on a remote computing device coupled thereto, know of feelings regarding tightness in the chest, and the eyewear could respond as above.

In one embodiment, to identify confusion, the digital assistant can engage in dialogues with the user. Through the dialogue, the digital assistant could spot changes in patterns in the user's spoken language, which could infer confusion in the user. The digital assistant could also ask the user simple questions, such as “What year is it?” If the user previously can respond correctly, based on mistakes in the new responses, the digital assistant could identify that the user is confused.

In one embodiment, bluish lips or face could be identified based on image recognition and processing. One or more cameras at the eyewear could periodically take images of the user. The images could be analyzed accordingly to identify color change, such as bluish lips or face. Such information can be combined with the user's oxygen level to determine if medical personnel needs to be immediately notified.

In one embodiment, the eyewear's database could include guidelines from the government regarding a particular disease, such as COVID-19. According to, for example, one set of government rules, 10 days since symptoms of COVID-19 first appeared, plus 24 hours with no fever without the use of fever-reducing medications, and other symptoms of COVID-19 also improving (other than symptoms regarding loss of taste and smell), would allow the user to be freed from quarantine. The eyewear could be used to identify many of the above parameters. In addition, the digital assistant within the eyewear, or provided by an application operating on a remote computing device coupled to the eyewear, could also confirm with the user regarding the above parameters. If all the parameters are satisfied, the eyewear could notify the user that the user is allowed to end isolation.

In one embodiment, a database available to the eyewear, or an application operating on a remote computing device coupled thereto, could include some standard recovery information regarding the particular disease. For example, if most people having the disease feel tired for a number of weeks, a digital assistant could let the user know so the user is following a normal recovery pattern and thus that the user should not feel discouraged or depressed if the user still feels tired several days after the disease has seemingly gone away. Such interactions could be automatic by the digital assistance or initiated by the user.

Various other embodiments for electronic eyewear can offer the following features which are suitable, for example, for eyewear applicable to COVID-19, hereinafter, the COVID-19 eyewear. In one embodiment, the COVID-19 eyewear can perform a number of different measurements, which can indicate the likelihood that a user might be infected with COVID. In another embodiment, the COVID-19 eyewear can also help a user fight COVID if the user has contracted the virus.

### Resting Heart Rate, Heart Rate Variability, and Oxygen-Level

The onset of COVID can be signaled by increasing resting heart rate and decreasing resting heart rate variability. Heart rate variability is a measure of variations in time intervals between heartbeats, or how “uneven” one's heartbeat is.

The onset of the disease can also lead to decreased oxygen-level. Oxygen level can also provide an indication of the health of the user's lungs.

The above measurements can be done, for example, by a pulse oximeter. In one embodiment, a pulse oximeter can be implemented using, for example, red LED, near-infrared LED and photodiode. A pulse oximeter can be at a nose pad of the eyewear.

Different examples of heart rate measurements via an electronic eyewear can be found in U.S. Pat. No. 7,677,723, “Eyeglasses with a heart rate monitor,” which is incorporated herein by reference.

### Temperature Measurement

The onset of the COVID-19 can lead to increasing body temperature.

Temperature can be measured, for example, by a near-infrared photodiode in the eyewear pointing at the skin of the user, such as pointing at the user's forehead or an eye of the user. Another approach is to use a thermistor touching the skin of the user, such as at a nose pad of the eyewear.

### Activity Level Measurement

The onset of the disease can lead to fatigue or decreasing activity level.

Different examples of activity monitoring via an electronic eyewear can be found in U.S. Pat. No. 7,255,437, “Eyeglasses with Activity Monitoring,” which is incorporated herein by reference.

### Breathing Rate Measurement

The onset of the disease can lead to an increased breathing rate.

Breathing rate can be measured, for example, based on measuring at-rest heart rate variability. When the user is at rest, as the user inhales, his heart rate increases, and as the user exhales, the heart rate decreases. By monitoring the beat-to-beat changes in the user's heart rate, the eyewear could identify the breathing rate of the user.

### Shortness of Breath Measurement

The disease can lead to shortness of breath or an increase in shortness of breath.

Shortness of breath can be measured, for example, by measuring the breathing rate of the user. One way to identify if there is shortness of breath or an increase in shortness of breath is to first determine the normal at-rest breathing rate of the user. If that is not available, one approach can use the average breathing rate of a person at the age of the user. Then the eyewear could compare the current at-rest breathing rate of the user with the normal at-rest breathing rate of the user, to evaluate whether the user has shortness of breath or an increase in shortness of breath.

### Coughing Frequency Measurement

The onset of the disease can lead to coughing, particularly dry coughing.

Coughing can be measured, for example, by using one or more microphones in the eyewear. In one approach, there could be a database including coughing and other types of sound made by humans. The audio sound by the user can be compared to the sound in the database to determine if the audio sound by the user is a cough. The eyewear could also record the coughing and determine its frequency over time.

Further, coughing can cause a brief increase in heart rate. One way to detect coughing can be by measuring temporary increases in the user's at-rest heart rate, with the increase not sustained.

Such changes in heart rate could be correlated with the coughing signals (i.e., audio signals) captured by the microphone(s). This correlation could help to increase the accuracy of coughing detection.

### Stuffy Nose Measurement

One way to determine if a user has a stuffy nose can be based on measuring the voice of the user. For example, if the user has stuffy nose, the frequency spectrum of the user's voice can be changed. The fundamental frequency of the user's voice may remain the same since the fundamental frequency is typically driven by the vibration of the vocal cords. However, the frequency spectrum of the voice can change since the spectrum can depend on the resonance characteristics of the vocal tract, which can include the nasal cavity. So, the voice may have a lower pitch due to the stuffy nose. With a lower pitch, the average frequency of the user's voice may be lower.

One embodiment to determine for stuffy nose is to first identify the average frequency of the voice of the user in his normal state or typical state. If the average frequency of the user's voice has been lowered for a duration of time, such as over a day, the embodiment can infer that there is a higher likelihood that the user has a stuffy nose. Another approach is to identify the frequency spectrum of the user in his typical state, or identify his average frequency spectrum over a period of time. An inference can be made based on the current frequency spectrum of the user's voice being shifted downward, or based on the current average frequency spectrum being shifted downward over a duration of time.

### Keep track of Where the User Has Been

The risk of getting COVID increases if the user has been to a COVID-19 hot spot.

The user's eyewear could include GPS or other location identifying capabilities, such as based on cellular or other wireless signals. The eyewear could keep track of its location as a function of time, such as for the past three weeks.

### Distance Measurement

The chance of getting COVID-19 can depend on how close the user has been with others, in particular, with those having COVID-19. Since COVID-19 patients can be asymptomatic, it would be useful to identify and keep track of the distances of people approaching or in contact with the user, and how many have approached the user across time, such as in the past few weeks.

### If Both Parties Have COVID-19 Eyewear

Distance measurements can be done, for example, based on the user and another person both wearing COVID-19 eyewear, or one wearing COVID-19 eyewear and the other wearing or carrying a corresponding wireless mobile device.

In one embodiment, the method can be based on a short-range wireless network, such as Bluetooth network.

The user's eyewear could broadcast a Bluetooth message (with an identifier) as needed or periodically, such as every 30 seconds, at a certain power level. If an approaching person is within the reception range of the signals, that person's eyewear (or wireless mobile device) could send a response automatically. When the user's eyewear gets the response, the user's eyewear could increase the frequency of broadcasting Bluetooth messages, such as every 50 milliseconds, but at different power levels. For example, after getting the response, the user's eyewear could send out another Bluetooth message at a lower level, such as ½ the power of the previous signals. This process of reducing the power level could continue until the user's eyewear does not get a response. At that point, the signal-to-noise ratio of the Bluetooth message received by the person's eyewear has reached a preset low-threshold level. Based on the transmitted power by the user's eyewear at that point, the distance between the two eyewear can be calculated.

If the person's eyewear is within a certain distance, such as two meters, from the user's eyewear, the user's eyewear could issue an alert, such as emitting an audible beep. The eyewear could alternatively or additionally record the incident, including denoting the location, time, and/or identifying other data concerning the person, or the person's COVID-19 eyewear or wireless mobile device.

### If Only the User Has COVID-19 Eyewear

Distance measurement can also be done if an approaching person does not wear a COVID-19 eyewear or a corresponding wireless mobile device. One approach is by using, for example, two visual-light cameras in the user's eyewear.

The two visual-light cameras can be positioned apart from each other in the user's eyewear, such as one at each of the extended end pieces of the eyewear. For example, the visual-light cameras can be CCD chips. In one embodiment, resolution of the images captured could be low, such as 100 pixels by 100 pixels.

Based on image processing, the user's eyewear could identify an approaching person, such as by visually detecting the head of the approaching person. The images of the person's head by the two visual-light cameras can be offset from each other, because they measure the head from different positions. The distance of the head from the visual-light cameras (or the user's eyewear) can be calculated based on the concept of parallax and the difference between the images from the cameras.

The eyewear could also include a near-infrared camera, which can be at the bridge of the eyewear, or at another area of the eyewear, such as at an extended end piece area. Assuming an object is approaching the user, the near-infrared camera can roughly estimate the temperature of the object. If the temperature is roughly within the range of the temperature of a human, the two visual-light cameras can then take images to perform distance measurements. Alternatively, a visual-light camera can determine a person is nearby, and then the near-infrared camera can be used to acquire temperature of the nearby person.

In the above approach, instead of visual-light camera, the user's eyewear could have near-infrared camera. For example, instead of two visual-light cameras and a near-infrared camera, the user's eyewear could have two near-infrared cameras instead. The near-infrared cameras can provide both rough temperature measurements and distance measurements.

### Temperature of an Approaching Person Measurement

It is important to keep track of an approaching person who has COVID. One way to determine if an approaching person has COVID is by measuring the person's temperature.

Temperature of an approaching person can be measured, for example, by a near-infrared camera and a visual-light camera in the user's eyewear. Both cameras can measure the forehead area or eye area of an approaching person. The temperature of the person can be estimated by the difference of the outputs from the two cameras. Multiple readings could be taken, and the temperature of the approaching person can be based on averaging the readings.

In one approach, the eyewear does not perform the above measurements until the approaching person is within a few meters from the user, such as two meters from the user.

### Contact Tracing

One approach for contact tracing depends on the approaching person also wearing a COVID-19 eyewear. The two pairs of eyewear could ping each other wirelessly and exchange identification signals if they are within a certain distance, such as two meters, from each other for more than a preset amount of time, such as 1 minute. The process can be via Bluetooth communications or other short-range wireless signals. The user's eyewear can keep a record of the identity of the approaching person.

Another way can depend on the eyewear being aware of a database of people infected by the disease. For example, the user's eyewear can keep track of the received identification signals from the approaching person, and check them against the database of individuals known to be infected. If the results are affirmative, the user's eyewear can keep a record of the encounter, and/or the user can be alerted.

### Database with COVID-Specific Information

Typically, each disease has its signature. For example, it is not uncommon during the very early phase, a COVID patient has an elevated temperature and heart rate, but reduced activity level and heart rate variability.

The eyewear could include (or have wireless access to) a database storing disease information, including typical symptoms of COVID, common conditions of those infected by the disease, typically recovery process, and other known information regarding the disease, such as from CDC (Centers for Disease Control and Prevention) guidelines.

Such disease-specific information could be coupled to measurements by the eyewear to provide observations and/or recommendations for the user and/or health-care providers regarding the user's conditions.

### Risk Identification

Based on some or all of the above measurements and information in the database regarding the disease, the eyewear could determine the likelihood of the user being infected by COVID-19. If the likelihood is high, the eyewear could suggest that the user should consider moving to a safer area and/or self-quarantine to avoid infecting others. The eyewear could also suggest that the user should schedule a COVID test. Further, the eyewear could provide a list of places closest to the user that provide such test.

A number of embodiments have been described regarding checking for user attributes to determine if the user has a higher chance of contracting a disease. In another embodiment, attributes of a user could indicate that the user has a lower chance to have the disease. For example, if stuffy nose is not a typical symptom of a disease and if the user has a stuffy nose, this would reduce the chance that the user has the disease.

### The User Infected by COVID-19

In one embodiment, the eyewear can also help the user if the user has been infected by COVID-19.

### Digital Assistant

A digital assistant could be incorporated in (or wirelessly coupled to) the eyewear to help the user. The digital assistant could be based on voice recognition, and could be tailored for the disease, such as designed based on recognizing information in the database regarding the disease. In other words, the digital assistant does not have to be designed to respond to all types of questions. Instead, the digital assistant could be designed to respond to queries regarding that disease.

The digital assistant can provide gentle reminders to the user, such as be regularly hydrated, or getting out to get some sun during the day. As another example, if the user has increased coughing, but at a much lower than critical level, the eyewear could identify such conditions, and the digital assistant could suggest to the user to drink a cup of warm water with honey.

The user could ask the digital assistant questions. If the database has answers to the questions, the digital assistant could respond accordingly. For example, the user could ask the digital assistant how many days her family member should be quarantined, or whether the user can end isolation. One set of government rules to end isolation could be 10 days since symptoms of COVID-19 first appeared, plus 24 hours with no fever without the use of fever-reducing medications, and other symptoms of COVID-19 also improving (other than symptoms regarding loss of taste and smell). The eyewear could identify many of the above parameters and confirm with the user. If all the parameters are satisfied, the eyewear could notify the user that the user may end isolation.

The eyewear could periodically give the user some moral support. For example, the digital assistant could ask the user if the user wants to hear a song that the digital assistant knows the user likes. Such information can be based on information in the user profile previously provided by the user. If the user's response is yes, the digital assistant could have the eyewear play the song for the user.

The eyewear could keep track of the user's different attributes, as measured by different detectors in the eyewear. The eyewear could compare the tracked attributes with information in the database. The eyewear can inform the user how closely they are correlating to symptoms of COVID-19 or other disease. If the user's conditions have worsened, the eyewear could suggest to the user to contact medical personnel.

If conditions monitored have significantly worsened, the eyewear could alert medical personnel directly. In the notification to medical personnel, the eyewear could provide tracked data regarding the user as well. For example, if the user's oxygen level has gone down significantly, the eyewear could prompt the user to call medical personnel. Or the eyewear could alert medical personnel directly.

The monitored information could indicate that the user is recovering. For example, if the user's heart rate variability is increasing, it is a sign that the user could be recovering.

### Recovered

In one example, the database in (or wirelessly coupled to) the eyewear could include some standard information of those who have recovered from the disease. For example, if most people feel tired for at least a week afterwards, the digital assistant could let the user know so the user would not feel discouraged or depressed if the user still feels tired days after her temperature has subsided. Such interactions can be initiated by the user. The eyewear can inform the user how closely they are correlating to normal recovery conditions or symptoms.

### “Long Haul” COVID-19

A significant number of patients still can be sick for months, not just weeks, after diagnosis of the disease. This can be “long haul” COVID-19. Currently, it may not be clear why that happens, let alone how to treat it.

The eyewear could keep track of major factors and/or monitored data of the patients through this long period to help understand and hopefully cure such patients.

A number of solutions above could be implemented in a smart phone wirelessly linked to the user's eyewear, such as via Bluetooth. For example, the database and the digital assistant in the eyewear could be in the phone. In another example, data captured by the eyewear also could be stored in a smart phone. The eyewear or phone can also be connected to a cloud-based server. User information can be stored in the server, with proper security measure, such as encryption.

### Additional Aspects

The eyewear can be a fit-over or safety glasses. The eyewear could be goggles, which could incorporate disposable masks, and can protect all of the user's mucosal surfaces.

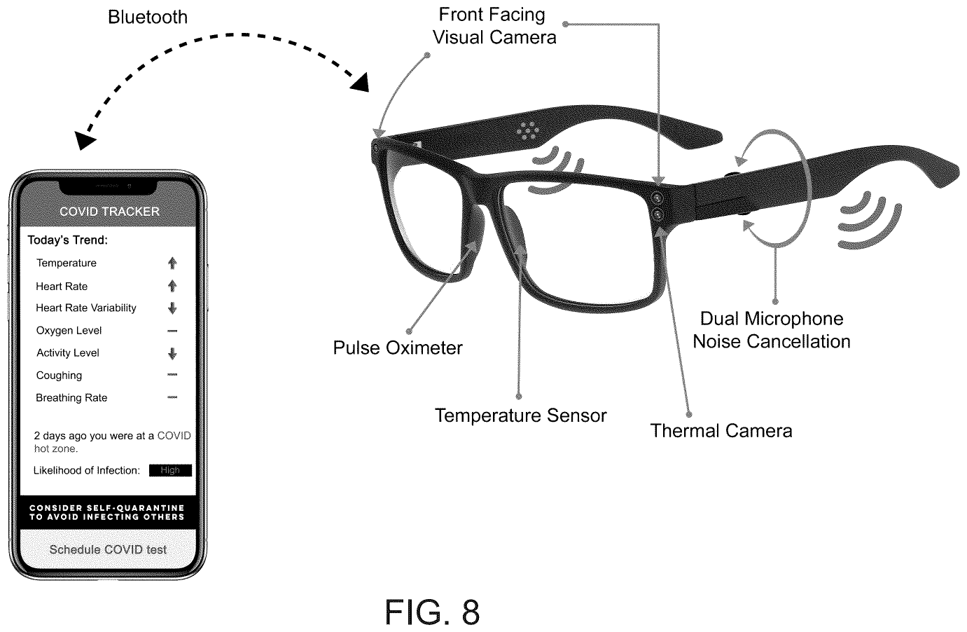

FIG. 8 is a diagram illustrating a pair of eyeglasses capable of being wirelessly coupled to a mobile phone, according to one embodiment. The eyewear can include a pulse oximeter at a nose pad of the eyewear, and can include a temperature sensor at another nose pad of the eyewear. The eyewear can include a motion sensor, with its electronics embedded in the eyewear (e.g., in an arm or temple of the eyewear). The eyewear can include two or more microphones, such as in a temple of the eyewear. The microphones can be directional microphones, and can be used for to provide noise cancellation. In one implementation, one microphone can point towards the mouth direction of the user, when the eyewear is worn, and the other microphone can be point away from the mouth direction of the user, such as pointing upwards when the eyewear is worn. The eyewear can include electronics to operate in a short-range wireless network, such as Bluetooth or Wi-Fi network. The eyewear can include two front-facing visual cameras, and a thermal camera. The thermal camera can be a near-infrared camera. The eyewear can include one or more speakers, which can be at least partially embedded in a temple of the eyewear. With two or more speakers, stereo sound can be provided for the user. The eyewear could include acoustic chambers in the temples so sound could be more directional towards the ears of the user.

The eyewear can be wirelessly coupled to a mobile device, such as a smart phone. Different functions can be performed in the mobile device, instead of in the eyewear. For example, a location detector (e.g., GPS receiver) can be in the mobile device. Additional electronics can be in the eyewear or in the mobile device. For example, electronics to identify location can be in the mobile device. At least a portion of computer programming code for a digital assistant can be in the mobile device. At least a portion of a database with information regarding COVID can be in the mobile device. The mobile device can include a display showing a number of attributes regarding the user, such as with respect to the disease. For example, the user's temperature has gone up, while the oxygen level has not changed much. Based on the tracked information and based on information regarding COVID, the user can be recommended to consider self-quarantine to avoid infecting others and to schedule for a COVID test.

In one embodiment, the eyewear can reduce its power consumption and cost by having a number of components (such as activity monitor and location detector) moved to, with their corresponding operations performed by, a mobile phone wirelessly coupled to the eyewear. The wireless coupling could be via short-range communication mechanism, such as Bluetooth. The embodiment can also assume other parties, such as a person approaching the user, have their corresponding mobile phones. In the embodiment, contact tracing, distance measurements, and temperature determination of other people, such as an approaching person, can be performed by mobile phones. For example, the user's mobile phone could get the temperature of the approaching person from that person's mobile phone, assuming that person's mobile phone has the information. In the embodiment, the eyewear can include a controller, a storage device, a pulse oximeter and a temperature sensor to monitor the user, noise-cancellation microphones, and a speaker.

The eyewear also can include an activity monitor to monitor the user, such as whether the user has fallen. In one implementation, the eyewear can be provided as a pair of fit-over glasses. The user's eyewear can be a pair of auxiliary glasses, which can include extended end pieces. In one embodiment, the user's eyewear need not have temples or arms. In one embodiment, the eyewear includes a re-chargeable battery. In another embodiment, the eyewear includes a battery that is not rechargeable, and the eyewear can be for short-term use.

In another embodiment, the eyewear is not coupled to a mobile device. Instead, the eyewear can be coupled to a computing device via, for example, the Internet or the cloud. The coupling can be through cellular connection. The computing device could perform a number of functions previously performed by the mobile device.

In yet another embodiment, the eyewear can include a display to present information, instead of having to present information via a display at a mobile device, as shown in FIG. 8. In one embodiment, a display for an eyewear can be based on holograms, and does not have to include a physical screen to present information.

In the above, COVID-19 has been used as an example. In other embodiments, the eyewear could be applicable for other diseases.

For example, another disease could affect the user in different ways. During the early phase, the disease could have different symptoms, and the recovery process could be different.

The eyewear could include other types of sensors also. For example, the eyewear could have imaging sensors to measure conditions of the user's eyes, such as the user's iris and retina.

The eyewear could measure the user's skin temperature, which could be used to identify and/or manage stress.

The eyewear could include imaging sensors to measure the user's skin color.

The eyewear could include sweat sensor to analyze sweat from the user, such as the sweat's pH.

The eyewear could include electrodes, such as at the temples of the eyewear, to measure, for example, brain waves, rapid eye movement, and the speed of the rapid eye movement. For eye movement measurements, electrodes can be on the skin in the region of the eyes. For example, one electrode could be at a nose pad on the left side, and another around the left extended end piece touching the skin of the user.

The eyewear could listen to and analyze audio sounds made by the user. Based on the analysis, the eyewear could, for example, identify the tone of the sounds, and could infer the emotional state of the user. The digital assistant in the eyewear can confirm with the user if the inference is correct. Identifying the emotional state of the user can be useful to help the user recover from a disease.

The eyewear could include disease-specific information in its storage. Such information can be downloaded to the eyewear. Based on measurements by sensors at the eyewear and based on information regarding the disease in the eyewear's storage, the eyewear could determine the likelihood of the user being infected by the disease, and/or help the user fight the disease. Voice-recognition capability of the eyewear could also be trained based on the disease-specific information.

In various other embodiments, electronic eyewear can be goggles, fit-over glasses, sunglasses, safety glasses, training classes, etc. In at least one embodiment, the electronic eyewear can also include a face shield, which can be attached or integral.

In one embodiment, an electronic eyewear can keep track of its battery levels in the eyewear and provide battery alert when the battery goes below a certain level, such as 25% remaining.

Different embodiments can be applicable to companies where employees working for the company, such as within the company's building, campus or compound, would wear the electronic eyewear. Different embodiments can be applicable to schools where students attending classes in the schools would wear the electronic eyewear. Different embodiments can be implemented in goggles or eye shields, and the embodiments can incorporate a mask, which could protect all the mucosal surfaces. Different embodiments can be implemented in auxiliary eyewear, fit-over glasses, or safety glasses, which could fit over a pair of glasses a person is wearing.

Different embodiments can also include or interact with a digital assistant. The digital assistant, or at least a portion of it, could be in a smart phone wirelessly coupled to the electronic eyewear, via, for example, Bluetooth. To illustrate, for example, a microphone at the electronic eyewear could capture a message from the user. The eyewear could wirelessly transmit the message or a representation of the message to the smart phone. The phone could analyze the message, based on, for example, voice recognition software, and generate a response. Then, the smart phone could transmit the response back to the electronic eyewear. A speaker at the eyewear could present the response to the user of the electronic eyewear.

Additional details on electronic eyewear that can be used with the various embodiment of the eyewear described herein are found in U.S. Pat. No. 10,061,144, which hereby incorporated herein by reference, U.S. Pat. No. 10,777,048, which hereby incorporated herein by reference, and U.S. patent application Ser. No. 16/574,254 and U.S. patent application Ser. No. 16/821,810, which are both incorporated herein by reference.

The various aspects, features, embodiments or implementations of the invention described above can be used alone or in various combinations.

Some embodiments of the invention can, for example, include or couple to an application program for providing programming functionally. The application program can be implemented by software, hardware, or a combination of hardware and software. These embodiments can also be embodied as computer readable code on a computer readable medium. In one embodiment, the computer readable medium is non-transitory. The computer readable medium is any data storage device that can store data which can thereafter be read by a computer system. Examples of the computer readable medium generally include read-only memory and random-access memory. More specific examples of computer readable medium are tangible and include Flash memory, EEPROM memory, memory card, CD-ROM, DVD, hard drive, magnetic tape, and optical data storage device. The computer readable medium can also be distributed over network-coupled computer systems so that the computer readable code is stored and executed in a distributed fashion.

Numerous specific details are set forth in order to provide a thorough understanding of the present invention. However, it will become obvious to those skilled in the art that the invention may be practiced without these specific details. The description and representation herein are the common meanings used by those experienced or skilled in the art to most effectively convey the substance of their work to others skilled in the art. In other instances, well-known methods, procedures, components, and circuitry have not been described in detail to avoid unnecessarily obscuring aspects of the present invention.

In the foregoing description, reference to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments mutually exclusive of other embodiments. Further, the order of blocks in process flowcharts or diagrams representing one or more embodiments of the invention do not inherently indicate any particular order nor imply any limitations in the invention.

The many features and advantages of the present invention are apparent from the written description. Further, since numerous modifications and changes will readily occur to those skilled in the art, the invention should not be limited to the exact construction and operation as illustrated and described. Hence, all suitable modifications and equivalents may be resorted to as falling within the scope of the invention.

What is claimed is:

1. Eyewear of a user comprising:
a front portion with a left side and a right side;
a first side portion and a second side portion, one coupled to the left side of the front portion and the other coupled to the right side of the front portion;
a first microphone and a second microphone in the eyewear;
a controller in the eyewear coupled to at least the first microphone and the second microphone; and
a storage medium configured to store at least attributes of a plurality of sound, including an attribute of sound regarding cough,
wherein at least based on outputs from the first and the second microphone, and the data regarding sound in the storage medium, the controller is configured to determine if the user has coughed.

2. Eyewear as recited in claim 1 comprising a third microphone and a fourth microphone,
wherein the first side portion includes a top part and a bottom part,
wherein the second side portion includes a top part and a bottom part,
wherein the first microphone is configured to be in the top part of the first side portion,
wherein the second microphone is configured to be in the top part of the second portion,
wherein the third microphone is configured to be in the bottom part of the first side portion, and
wherein the fourth microphone is configured to be in the bottom part of the second side portion.

3. Eyewear as recited in claim 2,
wherein the first microphone is configured to be directly above the third microphone, and
wherein the second microphone is configured to be directly above the fourth microphone.

4. Eyewear as recited in claim 2 comprising:
a fifth microphone configured to be in the top part of the first side portion;
a sixth microphone configured to be in the top part of the second portion;
a seventh microphone configured to be in the bottom part of the first side portion; and
an eighth microphone configured to be in the bottom part of the second side portion,
wherein the first, second, third, and fourth microphone are closer to the front portion than the fifth, sixth, seventh, and eighth microphone.

5. Eyewear as recited in claim 4,
wherein the first microphone is configured to be directly above the third microphone,
wherein the second microphone is configured to be directly above the fourth microphone,
wherein the fifth microphone is configured to be directly above the seventh microphone, and
wherein the sixth microphone is configured to be directly above the eighth microphone.

6. Eyewear as recited in claim 1 wherein the controller is configured to determine an orientation of the eyewear.

7. Eyewear as recited in claim 6 comprising a gyroscope to help identify the orientation of the eyewear.

8. Eyewear as recited in claim 6, wherein the orientation of the eyewear is with respect to a preset orientation.

9. Eyewear as recited in claim 8, wherein the preset orientation is determined based on a calibration process.

10. Eyewear as recited in claim 8 comprising a compass to help identify the preset orientation.

11. Eyewear as recited in claim 1, wherein the controller is configured to generate a recommendation and output the recommendation to the user based on the determine.
